SEQUENCE LISTING (1 sequence)

SEQ ID NO: 1 - protein, 27 aa, unidentified
ALALEUALATHRPHETHRVALASNILE

(19) United States
(12) Patent Application Publication
(10) Pub. No.: US 2024/0303488 A1
(43) Pub. Date: Sep. 12, 2024

(54) METHOD AND SYSTEM FOR T-CELL RECEPTOR (TCR) ASSAY DESIGN

(71) Applicant: ImmunityBio, Inc., Culver City, CA (US)

(72) Inventors: Kamil Wnuk, Playa del Rey, CA (US); Jeremi Sudol, New York, NY (US)

(73) Assignee: ImmunityBio, Inc., Culver City, CA (US)

(21) Appl. No.: 18/601,946

(22) Filed: Mar. 11, 2024

Related U.S. Application Data
(60) Provisional application No. 63/489,413, filed on Mar. 9, 2023.

(51) Int. Cl. G06N 3/08; G16B 40/20

(52) U.S. Cl. CPC G06N 3/08; G16B 40/20

(57) ABSTRACT

A system and method of designing a T-cell receptor (TCR) assay includes the use of processor-based predictive modeling of an HLA binding classifier, T-cell response, sequencing T-cells, and TCR classifier/regression. Particularly, embodiments include feeding a representation of various peptides into a trained HLA binding classifier model configured to determine average binding predictions of overlapping peptides at each position of the viral or cancer protein. Based upon the average binding predictions, one or more peptide pools can be selected and fed into the T-cell response model, along with representative blood samples associated with a patient/patient population. Further, a sequenced resultant T-cell response can be used to detect T-cell response patterns. These detected patterns can be used to train the TCR classifier/regression model to predict or estimate a patient state. Ultimately, a primer can be designed using a detected minimum set of T-cell receptors for classifying or estimating the patient state.

### CROSS REFERENCE TO RELATED APPLICATIONS

This application claims priority to U.S. provisional application Ser. No. 63/489,413, entitled “Method and System for T-Cell Receptor (TCR) Assay Design,” filed on Mar. 9, 2023. This application relates to commonly owned U.S. patent application Ser. No. 17,670,385, entitled “HLA Clusters, Global Frequencies, and Binding Across SARS-COV-2 Variation,” filed Feb. 11, 2022, which is currently a co-pending application. These applications are incorporated herein by reference in their entirety.

### TECHNICAL FIELD

This disclosure relates generally to T-cell receptor (TCR) assays, and more specifically to using computer-based predictions to determine a TCR assay.

### REFERENCE TO SEQUENCE LISTING

This application contains a Sequence Listing in electronic format. The Sequence Listing file, titled N1077-10078US02_ST26.xml, was created on Mar. 8, 2023, and is 1,496 bytes in size. The information in electronic format of the Sequence Listing is incorporated herein by reference in its entirety.

### BACKGROUND

The human immune system comprises a network of biological processes that protect a person from bacteria, microbes, viruses, toxins, parasites, and diseases. The immune system detects and responds to a wide variety of pathogens, from viruses to cancer cells, distinguishing foreign objects from healthy tissue.

A virus comprises a fragment of DNA or RNA enveloped in a protective protein coating. When a virus or bacteria invades a person's body, it can replicate itself to cause an infection or disease. When the virus encounters a human cell, it can infect the cell by attaching itself to the cell wall and injecting its viral DNA into the cell. The viral DNA can cause the cell to reproduce new virus particles. In some cases, the viral DNA causes the infected cell to eventually die and burst, freeing the new virus particles. In other cases, the infected cell may remain alive but the viral DNA may cause viral particles to sprout off of the cell.

The immune system uses white blood cells to identify and destroy infected cells. The Major Histocompatibility Complex (MHC) (also known as the Human Leukocyte Antigen (HLA)) allows white blood cells to distinguish between healthy native cells and cells infected by external viruses or bacteria. MHC protein molecules mark cells for specific white blood cells (T lymphocytes or “T cells”) to detect viral infections. Specifically, the MHC protein molecules present fragments of proteins (peptides) belonging to an invading virus on the surface of the cell to highlight the infection. When a T cell recognizes the peptides on the surface of the infected cell, it can bind to the cell and either destroy it or attempt to heal it. In contrast, T cells do not typically react to healthy cells where the MHC protein molecules present own cell peptides (as known as self-peptides).

There are two major types of MHC protein molecules, class I and class II, which span the membrane of cells in an organism. In humans, these MHC protein molecules are encoded by several genes clustered in a region on chromosome 6. HLAs corresponding to MHC class I (referred to herein as “HLA-I”) present peptides from inside a cell. For example, if the cell is infected by a virus, the HLA system brings fragments of the virus to the surface of the cell so that the cell can be destroyed by the immune system. HLAs corresponding to MHC class II (referred to herein as “HLA-II”) present antigens from extracellular proteins outside of the cell to T-lymphocytes. These antigens stimulate the multiplication of T-helper cells (also called CD4+ T cells). CD4+ T cells play a major role in instigating and shaping adaptive immune responses, such as by stimulating antibody-producing B-cells to produce antibodies to that specific antigen. An epitope is a part of an antigen which can bind to an antibody and be recognized by the immune system.

Antibodies are Y-shaped proteins produced by white blood cells to aid in the elimination of a virus or help stave off the effects of a viral or bacterial infection. The ends of the forked Y-shaped branches of these proteins can respond and bind to a specific antigen (e.g., bacteria, virus, or toxin). When an antibody binds to the outer coat of a virus particle or the cell wall of a bacterium, it can stop virus or bacteria movement through a human cell wall. Alternatively, a large number of antibodies can bind to an antigen and signal to a complement system (i.e., a series of proteins manufactured in the liver) that the invader needs to be removed.

Vaccine shots can aid the body in generating its own antibodies to fight infections. Although many vaccines exist that can cure an ailment, coronaviruses and influenza are two examples of viral and bacterial infections that currently cannot be cured completely by vaccines. These types of viruses tend to mutate quickly and/or have too many different strains for complete protection in all instances. In some cases, vaccines for the coronavirus and influenza may be a good way to stave off the effects of a particular strain of a virus.

Given the latest global pandemic, medical researchers have focused on the rapid characterization of Severe Acute Respiratory Syndrome Corona Virus 2 (SARS-COV-2), the virus responsible for the coronavirus disease 2019 (COVID-19) global pandemic, to determine possible target proteins or peptides for generating a vaccine that can provide therapeutic treatment.

SARS-COV-2 has a single-stranded, positive-sense, RNA genome of approximately 30 kilobases (kb), which includes open reading frames encoding nonstructural replicase polyproteins and structural proteins, namely, spike (S), envelope (E), membrane (M), and nucleocapsid (N). The positive-sense genome can act as messenger RNA and can be directly translated into viral proteins by a host cell's ribosomes.

Throughout 2020, early results from research efforts pointed to highest HLA-I/-II binding recognition from SARS-COV-2 spike (S) and nucleocapsid (N) proteins. Some researchers observed that SARS-COV-2 S and N proteins have the most candidate T & B cell epitopes. This research used reference “Wuhan-Hu-1” viral strain proteins and was based on conserved epitopes from SARS-COV (the 2003 SARS virus) and SARS-COV-2 predictions (determined using NetMHC4.0pan) across 12 HLA-I alleles. T-cell epitopes with high sequence identity to SARS-COV-2 were independently identified by both methods.

Other researchers have observed that genetic variability across the three MHC class I genes (HLA A, B, and C) may affect susceptibility to and severity of SARS-COV-2. They executed an in silico analysis of viral peptide-MHC class I binding affinity across 145 HLA-A, -B, and -C genotypes for all SARS-COV-2 peptides, and explored the potential for cross-protective immunity conferred by prior exposure to four common human coronaviruses. The analysis showed 48 highly conserved amino acid sequence spans across 34 distinct coronaviruses (ORF1ab, S, E, M, and N proteins), and 56 HLAs that had no affinity for conserved peptides. It also showed that the SARS-COV-2 proteome is successfully sampled and presented by a diversity of HLA alleles. However, HLA-B*46:01 had the fewest predicted binding peptides for SARS-COV-2, suggesting individuals with this allele may be particularly vulnerable to COVID-19, as they were previously shown to be for SARS-CoV. Conversely, HLA-A*02:02, HLA-B*15:03, and HLA-C*12:03 showed the greatest capacity to present highly conserved SARS-COV-2 peptides that are shared among common human coronaviruses, suggesting it could enable cross-protective T-cell based immunity. Global distributions of HLA types were also reported with discussion on potential epidemiological ramifications in the setting of the COVID-19 pandemic.

Another strategy used by researchers is to use HLA-I and II predicted peptide “megapools” to identify circulating SARS-COV-2-specific CD8+ and CD4+ T cells in ˜70% and 100% of COVID-19 convalescent patients, respectively. CD4+ T cell responses to S proteins, the main target of most vaccine efforts, were robust and correlated with the magnitude of the anti-SARS-COV-2 IgG and IgA titers. The M, S, and N proteins each accounted for 11%-27% of the total CD4+ response, with additional responses commonly targeting nsp3, nsp4, ORF3a, and ORF8, among others. For CD8+ T cells, S and M proteins were recognized, with at least eight SARS-COV-2 ORFs targeted. Additionally, SARS-COV-2-reactive CD4+ T cells were detected in ˜40%-60% of unexposed individuals, suggesting cross-reactive T cell recognition between circulating “common cold” coronaviruses and SARS-COV-2.

One proposed SARS-COV-2 vaccine design concept is based on the identification of highly conserved regions of the viral genome and newly acquired adaptations, both predicted to generate epitopes presented on MHC class I and II across the vast majority of the human population. Using this concept, genomic regions that generate highly dissimilar peptides from the human proteome are prioritized. These are also predicted to produce B cell epitopes. Researchers have proposed sixty-five 33-mer peptide sequences predicted to drive long-term immunity for most people, a subset of which could be tested using DNA or mRNA delivery strategies. These included peptides that are contained within evolutionarily divergent regions of the spike (S) protein reported to increase infectivity through increased binding to the ACE2 receptor and within a newly evolved furin cleavage site thought to increase membrane fusion.

As a backdrop to these efforts, Artificial Neural Networks (ANNs), such as Recurrent Neural Networks (RNNs), have been used successfully in recent years for many tasks involving sequential data, where the RNN must find connections between long input and output sequences, such as for binding predictions between full peptide and HLA protein sequences. Attention mechanisms that enable improved performance in many tasks are an integral part of modern RNN networks. An attention mechanism can allow the RNN to focus on certain parts of an input sequence when predicting a certain part of an output sequence, enabling easier learning and higher quality predictions.

So far, however, current techniques have yielded limited information in terms of how HLA-I/II binding of SARS-COV-2 proteins can vary across viral strains and world populations. Particularly, current techniques have not provided sufficient insight into the nexus between HLA-I/II clusters, global frequencies, and binding across SARS-COV-2 variation. For example, vaccine researchers have yet to find effective techniques that minimize the chances of missing clusters of uniquely functioning HLAs in the quest for SARS-COV-2 vaccines or therapeutic treatments. Without techniques that yield such information, it has been difficult for medical researchers to achieve the validation and implementation of vaccine or therapeutic treatment concepts that specifically target vulnerabilities of SARS-COV-2 and engage a robust adaptive immune response in the vast majority of the world population. Current techniques also provide limited options for precisely tracking the healing progress of a patient or predicting the advancement of a SARS-COV-2 or other viral infection.

### SUMMARY

In response to the challenges described above, systems, methods and articles of manufacture for designing a T-cell receptor (TCR) assay for classifying or estimating a patient state are described herein.

In an embodiment, a system and method for designing a TCR assay that classifies and/or estimates the patient state is provided. One system of designing the TCR assay includes the use of processor-based predictive modeling of an HLA binding classifier, T-cell response, sequencing T-cells, and TCR classifier/regression. Particularly for some embodiments, the method may include training an Artificial Neural Network (ANN), such as a Convolutional Neural Network (CNN) or Recurrent Neural Networks (RNN), that defines a Pan-Human Leukocyte Antigen (HLA) binding classifier model to determine average binding predictions of overlapping peptides at each position of the viral or cancer protein. A plurality of inputs representing a plurality of peptides can be fed into the trained HLA binding classifier model. Based upon the average binding predictions, one or more peptide pools can be selected. Further, the one or more peptide pools and a plurality of inputs associated with a plurality of blood samples associated with a patient or patient population can be fed into T-cell response model. The resultant T-cell response can be sequenced using a sequencer. One or more T-cell response patterns can be detected from the sequenced T-cell response. The TCR classifier/regression model can be trained to predict or estimate a patient state based on the detected one or more T-cell response patterns. In some embodiments, a minimum set of T-cell receptors can be detected. Ultimately, a primer can be designed that defines a TCR assay using the detected minimum set of T-cell receptors for classifying or estimating the patient state.

In some embodiments, a system of TCR assay design is provided. A cloud-based TCR Assay system may include a processor coupled to a memory, a storage unit and a processor-based TCR assay module having an ANN model generator coupled to generate an HLA binding classifier model, a T-cell response model, and a TCR classifier/regression model. The HLA binding classifier model is configured to determine average binding predictions of overlapping peptides at each position of the viral or cancer protein. The TCR assay module may further include a peptide unit coupled to the HLA binding classifier model to feed a plurality of inputs representing a plurality of peptides into the trained HLA binding classifier model. Using the peptide unit, based upon the average binding predictions, one or more peptide pools can be selected. A sequencer may be included within the TCR assay module coupled to the T-cell Response model. The sequencer is designed to supply a plurality of inputs associated with a plurality of blood samples associated with a patient or patient population can be fed into T-cell response model. The sequencer is also configured to sequence the T-cell receptor response. One or more T-cell response patterns can be detected from the sequenced T-cell response. The TCR classifier/regression model can be configured to detect one or more T-cell response patterns. Further, the TCR classifier/regression model can be trained to predict or estimate a patient state based on the detected one or more T-cell response patterns. In some embodiments, TCR classifier/regression model can detect a minimum set of T-cell receptors for classifying or estimating the patient state. The TCR assay module may further include a primer agent to design a primer using a detected minimum set of T-cell receptors for classifying or estimating the patient state.

In some embodiments, a tangible, non-transitory, computer-readable medium having instructions thereon which, when executed by a processor, cause the processor to perform the TCR assay designing method described herein. In some embodiments, the method for designing a TCR assay is provided. Particularly, some embodiments may include training an ANN, such as a CNN or RNN, defining a HLA binding classifier model to determine average binding predictions of overlapping peptides at each position of the viral or cancer protein. A plurality of inputs representing a plurality of peptides can be fed into the trained HLA binding classifier model. Based upon the average binding predictions, one or more peptide pools can be selected. Further, the one or more peptide pools and a plurality of inputs associated with a plurality of blood samples associated with a patient or patient population can be fed into T-cell response model. The resultant T-cell response can be sequenced using a sequencer. One or more T-cell response patterns can be detected from the sequenced T-cell response. The TCR classifier/regression model can be trained to predict or estimate a patient state based on the detected one or more T-cell response patterns. In some embodiments, a minimum set of T-cell receptors can be detected. Ultimately, a primer can be designed that defines a TCR assay using the detected minimum set of T-cell receptors for classifying or estimating the patient state.

In some embodiments, the viral or cancer protein is encoded into variable-length peptides. The cancer or viral protein may comprise a SARS-COV-2 protein variant. The SARS-COV-2 protein variant may comprise a SARS-COV-2 nucleocapsid (N) protein variant. In other examples, the SARS-COV-2 protein variant comprises a SARS-COV-2 spike (S) protein variant.

In some embodiments, the determining of the average binding predictions includes classifying a peptide as a binder when an average binding prediction corresponding to the peptide satisfies a binding value threshold. The TCR assay design method may further include selecting the one or more peptide pools to focus on one or more of: a specific site, a hotspot, or a receptor-binding domain of the viral or cancer protein. In other embodiments, the one or more peptide pools may be selected to focus on multiple regions or hotspots of the viral or cancer protein. The one or more peptide pools may also be selected to focus on the entire viral or cancer protein. Further, the one or more peptide pools may be selected based on at least one of CD4 or CD8 T-cell interaction. In some embodiments, the one or more peptide pools may be selected based at least on the average binding predictions for the HLA-I functional groupings. Moreover, the one or more peptide pools may be selected based at least on the average binding predictions for the HLA-II functional groupings. The one or more peptide pools may also be selected based on areas of predicted binding frequency across the HLA-I and HLA-II functional groupings. The one or more peptide pools may be selected based on a pan-HLA binding prediction.

In some embodiments, the test for T cell response comprises at least one of the following: an enzyme-linked immunosorbent spot (ELISpot) assay test, a cytotoxic T Lymphocyte (CTL) assay test, and a DNA barcoded peptide-MHC (pMHC) multimers test. Further, the test for T-cell response may include testing a synthetic TCR assay for T-cell response.

In some embodiments, the synthetic TCR assay is designed to supplement T-cell response data for the patient or patient population. Further, the TCR assay can be used to classify or estimate a patient state. In some examples, the patient state comprises a determination of whether a patient has a medical condition. The patient state may also include an estimate of a medical outcome for a patient. Moreover, the patient state may comprise an estimate of a progression of a disease for a patient. In some embodiments, administering a therapeutic treatment to a patient based on the classified or estimated patient state may be included.

Other aspects and advantages of the embodiments will become apparent from the following detailed description taken in conjunction with the accompanying drawings which illustrate, by way of example, the principles of the described embodiments.

### BRIEF DESCRIPTION OF THE DRAWINGS

The described embodiments and the advantages thereof may best be understood by reference to the following description taken in conjunction with the accompanying drawings. These drawings in no way limit any changes in form and detail that may be made to the described embodiments by one so skilled in the art without departing from the spirit and scope of the described embodiments.

FIG. 1 is a block diagram of an exemplary network incorporating the systems and methods of designing a TCR assay, in accordance with some embodiments.

FIG. 2 is a block diagram of an exemplary system for TCR assay within the components of the exemplary network of FIG. 1, in accordance with some embodiments.

FIG. 3 is a block diagram of an exemplary TCR assay agent within the components of the exemplary network of FIG. 1, in accordance with some embodiments.

FIG. 4 is an exemplary flow diagram of a method for TCR assay design, in accordance with some embodiments.

FIG. 5 is an illustration showing an exemplary computing device which may implement the embodiments described herein.

### DETAILED DESCRIPTION

The following embodiments describe a system and method for designing a T-cell receptor (TCR) assay. It can be appreciated by one skilled in the art, that the embodiments may be practiced without some or all of these specific details. In other instances, well known process operations have not been described in detail in order not to unnecessarily obscure the embodiments.

In some embodiments, a system and method of designing a T-cell receptor (TCR) assay includes the use of processor-based predictive modeling of an HLA binding classifier, T-cell response, sequencing T-cells, and TCR classifier/regression. Particularly, some embodiments may include training an artificial neural network, such as a Convolutional Neural Network (CNN) or Recurrent Neural Networks (RNN), defining a pan-human leukocyte antigen (HLA) binding classifier model to determine average binding predictions of overlapping peptides at each position of the viral or cancer protein. A plurality of inputs representing a plurality of peptides can be fed into the trained HLA binding classifier model. Based upon the average binding predictions, selecting one or more peptide pools. Further, the one or more peptide pools and a plurality of inputs associated with a plurality of blood samples associated with a patient or patient population can be fed into T-cell response model. The resultant T-cell response can be sequenced using a sequencer. One or more T-cell response patterns can be detected from the sequenced T-cell response. A TCR classifier/regression model can be trained to predict or estimate a patient state based on the detected one or more T-cell response patterns, and a primer can be designed using a detected minimum set of T-cell receptors for classifying or estimating the patient state.

In some embodiments, the viral or cancer protein is encoded into variable-length peptides. The cancer or viral protein may comprise a SARS-COV-2 protein variant. The SARS-COV-2 protein variant may comprise a SARS-COV-2 nucleocapsid (N) protein variant. In other examples, the SARS-COV-2 protein variant comprises a SARS-COV-2 spike (S) protein variant.

In some embodiments, the determining of the average binding predictions includes classifying a peptide as a binder when an average binding prediction corresponding to the peptide satisfies a binding value threshold. The TCR assay design method may further include selecting the one or more peptide pools to focus on one or more of: a specific site, a hotspot, or a receptor-binding domain of the viral or cancer protein. In other embodiments, the one or more peptide pools may be selected to focus on multiple regions or hotspots of the viral or cancer protein. The one or more peptide pools may also be selected to focus on the entire viral or cancer protein. Further, the one or more peptide pools may be selected based on at least one of CD4 or CD8 T-cell interaction. In some embodiments, the one or more peptide pools may be selected based at least on the average binding predictions for the HLA-I functional groupings. Moreover, the one or more peptide pools may be selected based at least on the average binding predictions for the HLA-II functional groupings. The one or more peptide pools may also be selected based on areas of predicted binding frequency across the HLA-I and HLA-II functional groupings. The one or more peptide pools may be selected based on a pan-HLA binding prediction.

In some embodiments, the test for T cell response comprises at least one of the following: an enzyme-linked immunosorbent spot (ELISpot) assay test, a cytotoxic T Lymphocyte (CTL) assay test, and a DNA barcoded peptide-MHC (pMHC) multimers test. Further, the test for T-cell response may include testing a synthetic TCR assay for T-cell response.

In some embodiments, the synthetic TCR assay is designed to supplement T-cell response data for the patient or patient population. Further, the TCR assay can be used to classify or estimate a patient state. In some examples, the patient state comprises a determination of whether a patient has a medical condition. The patient state may also include an estimate of a medical outcome for a patient. Moreover, the patient state may comprise an estimate of a progression of a disease for a patient. In some embodiments, administering a therapeutic treatment to a patient based on the classified or estimated patient state may be included.

Advantageously, the system and method of designing a TCR assay enables tracking the progression of a viral infection within a patient. In particular, the method of TCR assay design can detect the progression of the infection based on T-cell response in view of the blood sample data associated with the patient or patient population.

In the following description, numerous details are set forth. It will be apparent, however, to one skilled in the art, that the present invention may be practiced without these specific details. In some instances, well-known structures and devices are shown in block diagram form, rather than in detail, to avoid obscuring the present invention.

Some portions of the descriptions which follow are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, as apparent from the following discussion, it is appreciated that throughout the description, discussions utilizing terms such as “providing,” “generating,” “installing,” “monitoring,” “enforcing,” “receiving,” “logging,” “intercepting”, or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Various embodiments also relate to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

Reference in the description to “one embodiment” or “an embodiment” means that a particular feature, structure, or characteristic described in connection with the embodiment is included in at least one embodiment of the invention. The phrase “in one embodiment” located in various places in this description does not necessarily refer to the same embodiment. Like reference numbers signify like elements throughout the description of the figures.

The various techniques described herein improve upon current techniques to provide insight into connections between HLA-I/II clusters, global frequencies, and binding regions across SARS-COV-2 variation. Particularly, the techniques are helpful for finding missing clusters of uniquely functioning HLAs in the quest for vaccines or antiviral therapeutic treatments. The techniques also provide for precise tracking of the healing progress of a patient and/or predicting the advancement of a viral infection. It should be appreciated that the various embodiments can be implemented in numerous ways, e.g., by a process, an apparatus, a system, a device, a method, or by a combination thereof. Several inventive embodiments are described below.

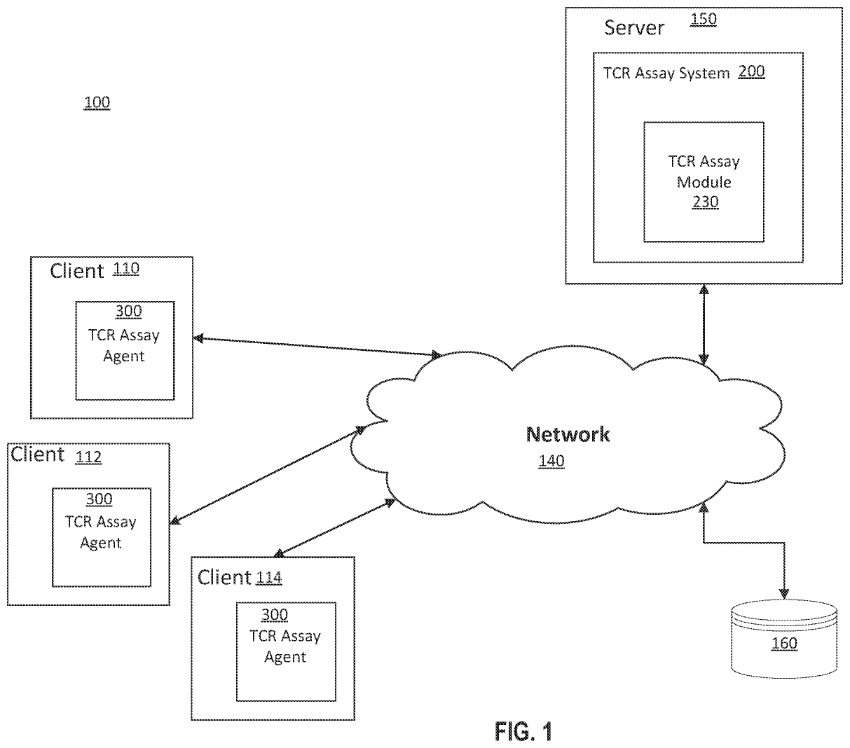

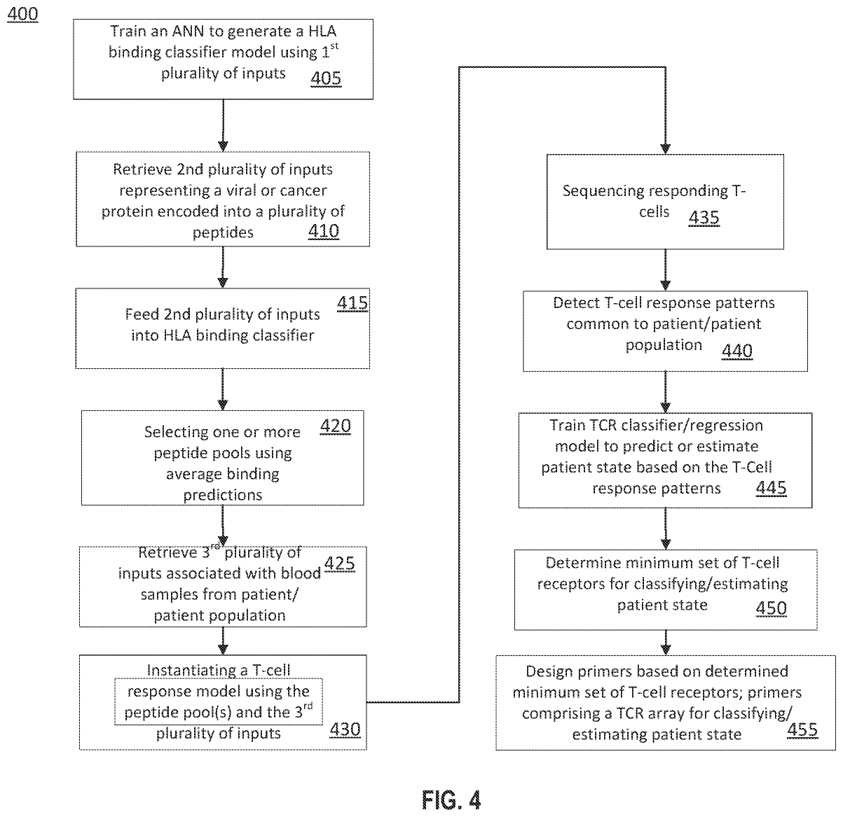

Referring to FIG. 1, an exemplary network incorporating the systems and methods of designing a T-cell receptor (TCR) assay is shown. As shown, the exemplary network architecture 100 may include at least one client node (computing devices) 110, 112, and 114, in communication with server 150 through network 140. As detailed above, all or a portion of network architecture 200 may perform and/or be a means for performing, either alone or in combination with other elements, one or more of the steps disclosed herein (such as one or more of the steps illustrated in FIG. 4). All or a portion of network architecture 100 may also be used to perform and/or be a means for performing other steps and features set forth in the instant disclosure. In one example, computing device 110 may be programmed with one or more of agents 300 (described in detail below). Additionally, or alternatively, server 150 may be programmed with one or more of modules 200. Although not shown, in various embodiments, the client node (110, 112, and 114) including TCR Assay agent 300 may be notebook computers, desktop computers, microprocessor-based or programmable consumer electronics, network appliances, mobile telephones, smart telephones, pagers, radio frequency (RF) devices, infrared (IR) devices, Personal Digital Assistants (PDAs), set-top boxes, cameras, integrated devices combining at least two of the preceding devices, and the like.

In some embodiments, TCR Assay agent 300, having peptide unit 340, sequencer 350, and primer agent 360, may serve as a device that communicates with the server 150 to perform the method of designing TCR Assays in real-time described more in detail below. In other embodiments, TCR Assay module 200 having a TCR assay design process utilizing predictive modeling may communicate with each client node 110, 112, and 114 and serve as the sole agent that performs the method of TCR Assay design method described herein. Client nodes 110, 112, and 114, server 150, and storage device 160 may reside on the same LAN, or on different LANs that may be coupled together through the Internet, but separated by firewalls, routers, and/or other network devices. In one embodiment, client nodes 110, 112, and 114 may be coupled to network 140 through a mobile communication network. In another embodiment, client nodes 110, 112, and 114, server 150, and storage device 160 may reside on different networks. In some embodiments, server 150 may reside in a cloud network. Although not shown, in various embodiments, client nodes 110, 112, and 114 may be notebook computers, desktop computers, microprocessor-based or programmable consumer electronics, network appliances, mobile telephones, smart telephones, pagers, radio frequency (RF) devices, infrared (IR) devices, Personal Digital Assistants (PDAs), set-top boxes, cameras, integrated devices combining at least two of the preceding devices, or the like. In some embodiments, each client node may comprise TCR assay module 230 operable entirely or partially to perform the TCR assay design in accordance with the method disclosed herein (client nodes 110, 112, and 114).

TCR assay server 150 may comprise a processor (not shown), memory (not shown), and TCR assay system 200, having the TCR assay module 230. In some embodiments, server 150 may comprise processing software instructions and/or hardware logic required for TCR assay design according to the embodiments described herein. Server 150 may provide remote cloud storage capabilities for call classifications, call filters, and various types of security policies associated, through storage device 160 coupled via network 140. In addition, server 150 may provide remote storage capabilities for ai model data, peptide data, T-cell response data and blood sample data. Further, server 150 may be coupled to one or more tape-out devices (not shown) or any other secondary datastore. As such, a database of patient profile data and user policy data may be stored within a local data store, remote disks, secondary data storage devices, or tape-outs devices (not shown). In some embodiments, client nodes 110, 112, and 114 may retrieve previous results relating to peptide pool, T-cell response, blood sample data from a remote datastore to a local data store 158. In other embodiments, the database of AI policies, prior TCR assay results, and the like may be stored locally on one or more of client nodes 110, 112, and 114 or server 150. For remote storage purposes, the local data storage unit 160 can be one or more centralized data repositories having mappings of respective associations between each fragment data and its location within remote storage devices. The local data store may represent a single or multiple data structures (databases, repositories, files, etc.) residing on one or more mass storage devices, such as magnetic or optical storage-based disks, tapes or hard drives. This local data store may be an internal component of server 150. In the alternative, local data store 160 also may couple externally to server 150 as shown in FIG. 1, or remotely through a network. Further, server 150 may communicate with the remote storage devices over a public or private network. Although not shown, in various embodiments, server 150 may be a notebook computer, desktop computer, microprocessor-based or programmable consumer electronics, network appliance, mobile telephone, smart telephone, radio frequency (RF) device, infrared (IR) device, Personal Digital Assistant (PDA), set-top box, an integrated device combining at least two of the preceding devices, and the like.

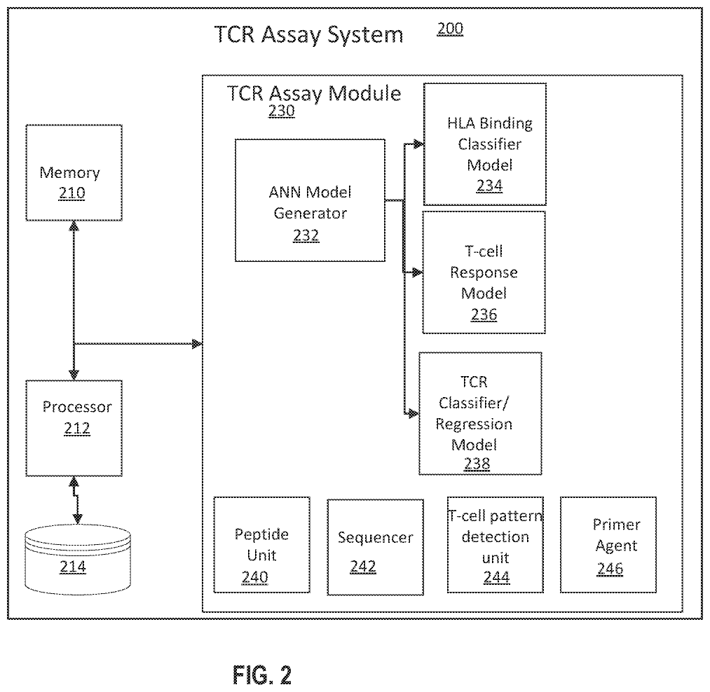

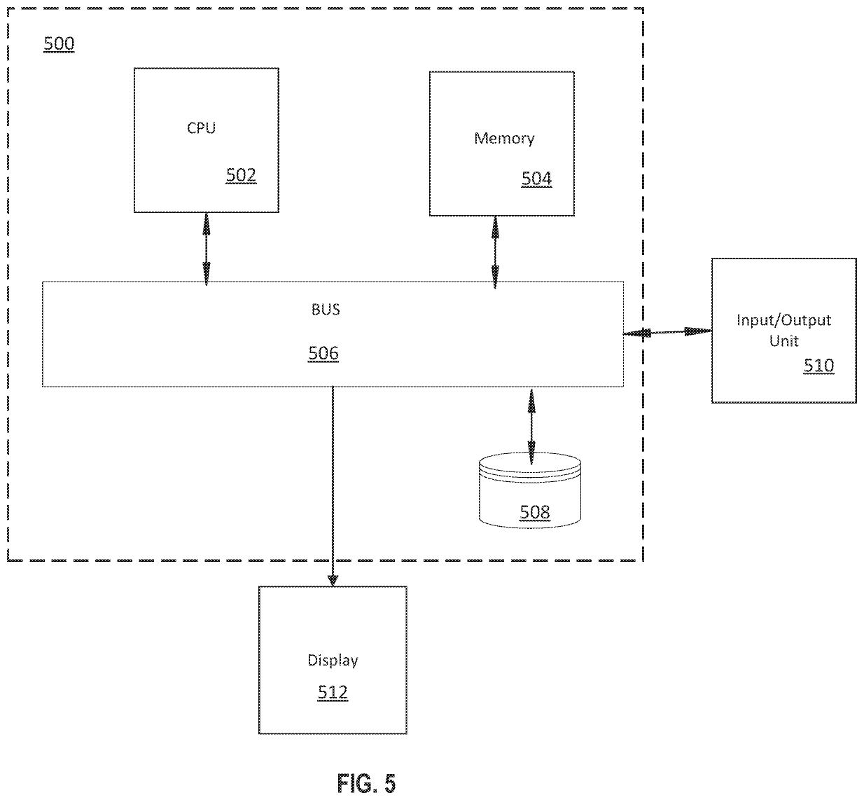

Client nodes 110, 112, and 114 generally represent any type or form of computing device or system, such as exemplary computing system 500 in FIG. 5. Similarly, server 150 generally represents computing devices or systems, such as application servers or database servers, configured to provide various database services and/or run certain software applications. Network 140 generally represents any telecommunication or computer network including, for example, an intranet, a WAN, a LAN, a PAN, or the Internet. For embodiment, client nodes 110, 112, and 114, and/or server 150 may include all or a portion of system 200 from FIG. 2.

In some embodiments, one or more storage devices (not shown) may be directly attached to server 150. Storage devices generally represent any type or form of storage device or medium capable of storing data and/or other computer-readable instructions. In certain embodiments, storage devices may represent Network-Attached Storage (NAS) devices configured to communicate with server 150 using various protocols, such as Network File System (NFS), Server Message Block (SMB), or Common Internet File System (CIFS)

Server 150 may also be connected to a Storage Area Network (SAN) fabric (not shown). The SAN fabric generally represents any type or form of computer network or architecture capable of facilitating communication between a plurality of storage devices. The SAN fabric may facilitate communication between server 150 and a plurality of storage devices (not shown) and/or an intelligent storage array (not shown). The SAN fabric may also facilitate, via network 140 and server 150, communication between client nodes 110, 112, and 114, and storage devices and/or an intelligent storage array in such a manner that devices 170(1)-(N) and array 180 appear as locally attached devices to client nodes 110, 112, and 114.

In certain embodiments, and with reference to exemplary computing system 500 of FIG. 5, a communication interface is used to provide connectivity between each client node 110, 112, and 114 and network 150. Client nodes 110, 112, and 114 are configured to access information from a database coupled to server 150 using, for example, a web browser or other client software. Such software may allow client nodes 110, 112, and 114 to access data hosted by server 150, local storage devices, remote storage devices, or intelligent storage array. Although FIG. 1 depicts the use of a network (such as the Internet) for exchanging data, the embodiments described and/or illustrated herein are not limited to the Internet or any particular network-based environment.

In at least one embodiment, all or a portion of one or more of the exemplary embodiments disclosed herein may be encoded as a computer program and loaded onto and executed by server 150, local storage devices, remote storage devices, or intelligent storage array, or any combination thereof. All or a portion of one or more of the exemplary embodiments disclosed herein may also be encoded as a computer program, stored in server 150, and distributed to one or more of client nodes 110, 112, and 114 via network 140.

One or more components of network architecture 100 may perform and/or be a means for performing, either alone or in combination with other elements, one or more steps of an exemplary method for TCR assay design. It is appreciated that the components of exemplary operating environment 100 are exemplary and more or fewer components may be present in various configurations. It is appreciated that operating environment may be part of a distributed computing environment, a cloud computing environment, a client server environment, and the like.

Referring to FIG. 2, an exemplary embodiment of TCR assay designing system 200 within the components of the exemplary network of FIG. 1 is shown. Exemplary system 200 may be implemented in a variety of ways. For example, all or a portion of exemplary system 200 may represent portions of exemplary system 100 in FIG. 1. As illustrated in this figure, exemplary system 200 may include memory 210, processor 212, and storage database 214. The system may include one or more TCR assay modules 230 for performing one or more tasks. For example, and as will be explained in greater detail below, TCR assay module 230 may include Artificial Intelligence Neural Network (ANN) generator 232 coupled to define an HLA binding classifier model 234, T-cell response model 236, and TCR classifier/regression model 238. TCR assay module 230 may further comprise a peptide unit 240, sequencer 242, primer agent 244, and T-cell pattern detection unit 246. Peptide unit 240 is configured to store and feed a plurality of encoded peptides into the trained HLA binding classifier model 234. Sequencer 242 is configured to sequence the identified responding T-cells. T-cell pattern detection unit 244 can detect the one or more T-cell response patterns common to the patient or patient population. Primer agent 246 is configured to design the one or more primers defining the TCR assay for classifying or estimating the patient state.

In operation, TCR assay module 230 may train an ANN defining pan-human leukocyte antigen (HLA) binding classifier model 234 using ANN model generator 232 within TCR assay module 230. Using a first plurality of inputs, trained HLA binding classifier model 234 is configured to determine average binding predictions of overlapping peptides at each position of the viral or cancer protein independently for each of a plurality of test HLAs comprising HLA-I and HLA-II functional groupings. Further, peptide unit 240 may retrieve from local or remote storage a second plurality of inputs that represent a viral or cancer protein encoded into a plurality of peptides. Peptide unit 240 may feed these inputs into trained HLA binding classifier model 234. HLA binding classifier model 234 may be configured to receive from peptide unit 240 a plurality of inputs representing the plurality of peptides. The peptide unit 240 can select one or more peptide pools from the plurality of peptides based on the average binding prediction derived from HLA binding classifier model 234. In some embodiments, the blood samples may be retrieved from one or more of the TCR assay agents 300 within the client nodes 110,112, or 114. ANN model generator 232 may generate T-cell response model 236 using the one or more peptide pools and the third plurality of inputs. The T-cell response model can be trained to predict peptides or protein fragments most likely to elicit a T-cell response based on a database of validated T-cell epitopes and peptides that failed to elicit a T-cell response. Predictions from the T-cell response model can further refine the peptide pool proposed by aggregated HLA-binding predictors to enhance precision of proposed epitopes. A sequencer 242 may sequence the identified from results of the test for T-cell response. T-cell pattern detection unit 244 can detect one or more T-cell response patterns common to the patient or patient population. ANN model generator 232 may generate TCR classifier/regression model 238 based at least on the one or more T-cell response patterns. T-cell response patterns may be identified by training a TCR classifier or regression model to discriminate TCR sequences that are specific to a disease or patient state, from TCR sequences that are general across patients not representative of the condition of interest. Alternatively, TCR patterns specific to patient conditions can be characterized by non-parametric means by identifying clusters of TCR sequences in a sequence embedding space unique to the condition of interest. The trained TCR classifier or regression model 238 can determine a minimum set of T-cell receptors for classifying or estimating the patient state. This selection can be made by selecting the top ranked TCR patterns according to the predictions scores of the condition specific TCR classifier, which appear across a broad set of patients. Primer agent 246 can design primers based on the determined minimum set of T-cell receptors, the primers defining the TCR assay for classifying or estimating the patient state.

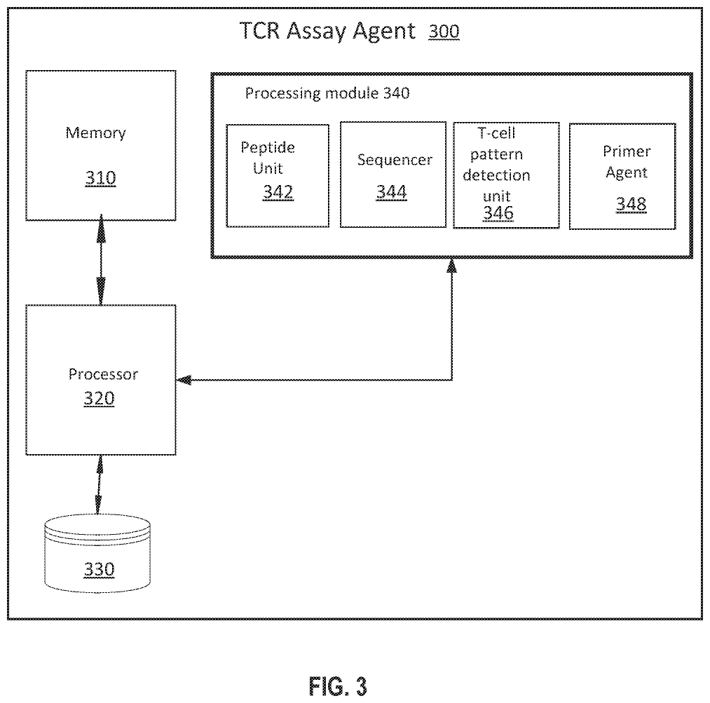

In some embodiments, the method for designing a T-cell receptor (TCR) assay may be implemented entirely within the TCR assay system 200 on server 150. In other embodiments, the method may be implemented using both the TCR assay agent 300 on the client node (110, 112, 114) and the TCR assay system 200 (to be described in more detail with respect to FIG. 3).

In some embodiments, the viral or cancer protein is encoded into variable-length peptides. The cancer or viral protein may comprise a SARS-COV-2 protein variant. The SARS-COV-2 protein variant may comprise a SARS-COV-2 nucleocapsid (N) protein variant. In other examples, the SARS-COV-2 protein variant comprises a SARS-COV-2 spike (S) protein variant.

In some embodiments, the determining of the average binding predictions includes classifying a peptide as a binder when an average binding prediction corresponding to the peptide satisfies a binding value threshold. The TCR assay design method may further include selecting the one or more peptide pools to focus on one or more of: a specific site, a hotspot, or a receptor-binding domain of the viral or cancer protein. In other embodiments, the one or more peptide pools may be selected to focus on multiple regions or hotspots of the viral or cancer protein. The one or more peptide pools may also be selected to focus on the entire viral or cancer protein. Further, the one or more peptide pools may be selected based on at least one of CD4 or CD8 T-cell interaction. In some embodiments, the one or more peptide pools may be selected based at least on the average binding predictions for the HLA-I functional groupings. Moreover, the one or more peptide pools may be selected based at least on the average binding predictions for the HLA-II functional groupings. The one or more peptide pools may also be selected based on areas of predicted binding frequency across the HLA-I and HLA-II functional groupings. The one or more peptide pools may be selected based on a pan-HLA binding prediction.

In some embodiments, the test for T cell response comprises at least one of the following: an enzyme-linked immunosorbent spot (ELISpot) assay test, a cytotoxic T Lymphocyte (CTL) assay test, and a DNA barcoded peptide-MHC (pMHC) multimers test. Further, the test for T-cell response may include testing a synthetic TCR assay for T-cell response.

In some embodiments, the synthetic TCR assay is designed to supplement T-cell response data for the patient or patient population. Further, the TCR assay can be used to classify or estimate a patient state. In some examples, the patient state comprises a determination of whether a patient has a medical condition. The patient state may also include an estimate of a medical outcome for a patient. Moreover, the patient state may comprise an estimate of a progression of a disease for a patient. In some embodiments, administering a therapeutic treatment to a patient based on the classified or estimated patient state may be included.

It is appreciated that the components of exemplary operating environment 100 are exemplary and more or fewer components may be present in various configurations. It is appreciated that operating environment may be part of a distributed computing environment, a cloud computing environment, a client server environment, and the like.

As used herein, the term module might describe a given unit of functionality that can be performed in accordance with one or more embodiments of the present invention. As used herein, a module might be implemented utilizing any form of hardware, software, or a combination thereof. For example, one or more processors, controllers, ASICs, PLAs, PALs, CPLDs, FPGAs, logical components, software routines or other mechanisms might be implemented to make up a module. In implementation, the various modules described herein might be implemented as discrete modules or the functions and features described can be shared in part or in total among one or more modules. In other words, as would be apparent to one of ordinary skill in the art after reading this description, the various features and functionality described herein may be implemented in any given application and can be implemented in one or more separate or shared modules in various combinations and permutations. Even though various features or elements of functionality may be individually described or claimed as separate modules, one of ordinary skill in the art will understand that these features and functionality can be shared among one or more common software and hardware elements, and such description shall not require or imply that separate hardware or software components are used to implement such features or functionality.

Referring to FIG. 3, an exemplary TCR assay agent 300 within the components of the exemplary network of FIG. 1 is shown. Exemplary agent 300 may be implemented in a variety of ways. For example, all or a portion of exemplary agent 300 may represent portions of exemplary system 100 in FIG. 1. More specifically, TCR assay agent 300 may include one or more of the components of the TCR assay module 200 for local processing of the method for designing a TCR assay described herein. In some embodiments, as illustrated in FIG. 3, exemplary agent 300 may include memory 310, processor 320, and storage database 330. The agent may include one or more processing modules 340 for performing one or more tasks. For example, and as will be explained in greater detail below, processing modules 340 may include a peptide unit 342, sequencer 344, and T-cell pattern detection unit 346 and primer agent 348. Similar to peptide unit 240, peptide unit 342 is configured to store and feed a plurality of encoded peptides into the trained HLA binding classifier model 234 of the TCR Assay Module 230 on server 150 (FIGS. 1 and 2). Sequencer 344 is configured to sequence T-cells identified from results of a test for T-cell response using a T-cell model generated by the ANN model generator 232 on server 150. T-cell pattern detection unit 346 can detect the one or more T-cell response patterns common to the patient or patient population based upon the T-cell model. In communication with the TCR Assay module 230, the primer agent 348 is configured to design the one or more primers defining the TCR assay for classifying or estimating the patient state, based upon a determined minimum set of T-cell receptors.

In operation, TCR assay module 230, in cooperation with the TCR agent 300, may train an ANN defining pan-human leukocyte antigen (HLA) binding classifier model 234 using ANN model generator 232 within TCR assay module 230. Using a first plurality of inputs from the peptide unit 342 and local store 330, trained HLA binding classifier model 234 is configured to determine average binding predictions of overlapping peptides at each position of the viral or cancer protein independently for each of a plurality of test HLAs comprising HLA-I and HLA-II functional groupings. Further, peptide unit 342 may retrieve from local or remote storage a second plurality of inputs that represent a viral or cancer protein encoded into a plurality of peptides. Peptide unit 342 may feed these inputs into trained HLA binding classifier model 234. Peptide unit 342 can select one or more peptide pools from the plurality of peptides based on the average binding prediction derived from HLA binding classifier model 234. As noted supra, in some embodiments, the blood samples may be retrieved from one or more of the TCR assay agents 300 within the client nodes 110, 112, or 114. ANN model generator 232 may generate T-cell response model 236 using the one or more peptide pools and the third plurality of inputs. Sequencer 344 may sequence the identified from results of the test for T-cell response. T-cell pattern detection unit 346 can detect one or more T-cell response patterns common to the patient or patient population. On the server 150, ANN model generator 232 may generate TCR classifier/regression model 238 based at least on the one or more T-cell response patterns. The trained TCR classifier or regression model 238 can determine a minimum set of T-cell receptors for classifying or estimating the patient state. In communication with the TCR classifier or regression model 238 on the server 150, primer agent 348 on any client node can design primers based on the determined minimum set of T-cell receptors, the primers defining the TCR assay for classifying or estimating the patient state.

FIG. 4 is an exemplary flow diagram of a method of designing a TCR assay in accordance with some embodiments. In action 405, an ANN is trained to generate an HLA binding classifier model using a first plurality of inputs. For example, ANN model generator 232 may train an ANN using a first plurality of inputs defining a pan-human leukocyte antigen (HLA) binding classifier model 234. Trained HLA binding classifier model 234 may be configured to determine average binding predictions of overlapping peptides at each position of the viral or cancer protein independently for each of a plurality of test HLAs comprising HLA-I and HLA-II functional groupings. Further, a second plurality of inputs may be retrieved, wherein the inputs represent a viral or cancer protein encoded into a plurality of peptides in action 410. For example, peptide unit 240 may retrieve from local or remote storage the second plurality of inputs that represent a viral or cancer protein encoded into a plurality of peptides. The method of designing a TCR assay may further include feeding the second plurality of inputs representing the plurality of peptides into the trained HLA binding classifier model in action 415. For example, HLA binding classifier model 234 may couple to the peptide unit 240 to receive a plurality of inputs representing the plurality of peptides. Further, the method of designing a TCR assay may include selecting, based at least on the average binding predictions, one or more peptide pools from the plurality of peptides in action 420. For example, peptide unit 240 can select one or more peptide pools from the plurality of peptides based on the average binding prediction derived from HLA binding classifier model 234. Furthermore, the method, in action 425, may include retrieving a third plurality of inputs associated with a plurality of blood samples, wherein the blood samples are representative of a patient or patient population. In some embodiments, the blood samples may be retrieved from one or more of the TCR assay agents 300 within the client nodes 110,112, or 114. In action 430, the method may include instantiating, by the ANN model generator, a T-cell response model using the one or more peptide pools and the third plurality of inputs. For example, the ANN model generator 232 may generate T-cell response model 236 using the one or more peptide pools and the third plurality of inputs. The method of designing a TCR assay may include sequencing, by a sequencer, responding T-cells identified from results of the test for T-cell response in action 435. For example, a sequencer 242 may sequence the identified from results of the test for T-cell response. The method may include detecting, based at least on data obtained from sequencing the responding T-cells, one or more T-cell response patterns common to the patient or patient population in an action 440. For example, the T-cell response model 236 can detect one or more T-cell response patterns common to the patient or patient population. In action 445, the method may include training, by the ANN model generator, a TCR classifier or regression model to predict or estimate a patient state using datasets based at least on the one or more T-cell response patterns. For example, ANN model generator 232 may generate TCR classifier/regression model 238 based at least on the one or more T-cell response patterns. Moreover, the method of designing a TCR assay may include determining a minimum set of T-cell receptors for classifying or estimating the patient state, using the trained TCR classifier or regression model, in action 450. For example, the trained TCR classifier or regression model can determine a minimum set of T-cell receptors for classifying or estimating the patient state. In action 455, the method may include designing primers based on the determined minimum set of T-cell receptors, the primers comprising a TCR assay for classifying or estimating the patient state. For example, the primer agent can design primers based on the determined minimum set of T-cell receptors, the primers comprising a TCR assay for classifying or estimating the patient state.

In another embodiment, the method of designing a TCR assay disclosed herein includes a process for producing an immunotherapeutic comprising antigen-reactive T-cells. In some aspects, the method comprises identifying neoepitope antigen-reactive T-cells. In some aspects, the method involves producing a population of neoepitope antigen reactive T-cells using one or more peptides that contain amino acid sequences identical to the patient-derived neoepitopes. PCT patent application WO/2022/086727 is hereby incorporated by reference herein . . .

Additional embodiments are described below.

(1) A method of designing a T-cell receptor (TCR) assay performed by a processor-based TCR assay module, the method comprising:
obtaining a first plurality of inputs representing a plurality of peptides;
training an Artificial Neural Network (ANN) defining a pan-human leukocyte antigen (HLA) binding classifier model using the first plurality of inputs, wherein the trained HLA binding classifier model is configured to determine average binding predictions of overlapping peptides at each position of the viral or cancer protein independently for each of a plurality of test HLAs comprising HLA-I and HLA-II functional groupings;
obtaining a second plurality of inputs representing a viral or cancer protein encoded into a plurality of peptides;
feeding the second plurality of inputs into the trained HLA binding classifier model, wherein the trained HLA binding classifier is configured to determine average binding predictions of overlapping peptides of the plurality of peptides;
selecting, based on the average binding predictions, one or more peptide pools from the plurality of peptides;
obtaining a third plurality of inputs associated with a plurality of blood samples, wherein the blood samples are representative of a patient or patient population;
instantiating, based on the one or more peptide pools and the third plurality of inputs, a T-cell response model; wherein the T-cell response model is trained to predict peptides and protein fragments associated with a high probability of eliciting T-cell response, based on validated T-cell epitopes and peptides failing to elicit a T-cell response;
sequencing, by a sequencer, responding T-cells identified based on T-cell response criteria;
detecting, based on data obtained from sequencing the responding T-cells, one or more T-cell response patterns common to the patient or patient population;
training a TCR classifier/regression model to predict or estimate a patient state using datasets based on the one or more T-cell response patterns;
determining, using the trained TCR classifier/regression model, a minimum set of T-cell receptors for classifying or estimating the patient state; and
designing one or more primers based on the determined minimum set of T-cell receptors, the one or more primers defining a TCR assay for classifying or estimating the patient state.

(2) The method of (1), wherein the training of the HLA binding classifier model comprises,
obtaining a plurality of test HLAs encoded into variable-length proteins, wherein the plurality of test HLAs comprises HLA-I and HLA-II functional groupings;
processing the encoded variable-length peptides corresponding to the viral protein and the variable-length proteins corresponding to the plurality of test HLAs using the classifier model such that, independently per test HLA, the classifier model is operable to determine an average binding prediction of overlapping peptides at each position of the viral protein;
independently per test HLA:
   mapping in aggregate average binding predictions to locations along the test viral protein such that peptide-HLA interaction is indicated;
   determining nearest max locations for the average binding predictions using a sliding window having a fixed length;
   determining top max regions by selecting the nearest max locations having average binding predictions within a top percentage of values;
   selecting peptides classified as binders that overlap the top max regions; and
   determining a pan-HLA max region, wherein the determining includes setting unselected locations to zero, calculating a mean along an HLA axis of the average binding prediction, and selecting pan-HLA maxima within a top percentage of values based on the mean;
independently for each of the HLA-I and HLA-II functional groupings:
   filtering the selected peptides classified as binders to identify candidate peptides that overlap the top max regions based on an aggregate of the pan-HLA max regions; and
   including one or more of the candidate peptides in an mRNA-based vaccine or therapeutic treatment for a patient.

(3) The method of any of (1)-(2), wherein the training of the TCR classifier/regression model to predict or estimate a patient state comprises:
differentiating TCR sequences specific to a patient state from general TCR sequences associated with patients not representative of a condition of interest associated with the patient state;
identifying T-cell response patterns based on the differentiation; and
generating the TCR classifier/regression model based on the identified T-cell response patterns.

(4) The method of any of (1)-(3), wherein the training of the TCR classifier/regression model to predict or estimate a patient state comprises:
differentiating TCR sequences specific to a patient state from general TCR sequences associated with patients not representative of a condition of interest associated with the patient state;
identifying, based on the differentiation, TCR sequences in a sequence embedding space associated with the condition of interest; and
generating the TCR classifier/regression model with the identified TCR sequences.

(5) The method of any of (1)-(4), wherein the determining of the minimum set of T-cell receptors comprises:
retrieving prediction scores of the trained TCR classifier/regression model for a plurality of patients; and
selecting, based on the retrieved prediction scores, one or more TCR patterns.

(6) The method of any of (1)-(5), wherein the method further comprises selecting the one or more peptide pools based on one or more of: a specific site, a hotspot, or a receptor-binding domain of the viral or cancer protein.

(7) The method of any of (1)-(6), wherein the method further comprises selecting the one or more peptide pools based on multiple regions or hotspots of the viral or cancer protein.

(8) The method of any of (1)-(7), wherein the method further comprises selecting the one or more peptide pools based on an entire viral or cancer protein.

(9) The method of any of (1)-(8), wherein the method further comprises selecting the one or more peptide pools based on at least one of CD4 T-cell interaction or CD8 T-cell interaction.

(10) The method of any of (1)-(9), wherein the method further comprises selecting the one or more peptide pools based on the average binding predictions for the HLA-I functional groupings.

(11) The method of any of (1)-(10), wherein the method further comprises selecting the one or more peptide pools based on the average binding predictions for the HLA-II functional groupings.

(12) The method of any of (1)-(11), wherein the method further comprises selecting the one or more peptide pools based on areas of predicted binding frequency across the HLA-I and HLA-II functional groupings.

(13) The method of any of (1)-(12), wherein the method further comprises selecting the one or more peptide pools based on a pan-HLA binding prediction.

(14) The method of any of (1)-(13), wherein the test for T cell response comprises at least one of the following: an enzyme-linked immunosorbent spot (ELISpot) assay test, a cytotoxic T Lymphocyte (CTL) assay test, and a DNA barcoded peptide-MHC (pMHC) multimers test.

(15) The method of any of (1)-(14), wherein the test for T-cell response further comprises testing a synthetic TCR assay for T-cell response.

(16) The method of (15), wherein the synthetic TCR assay is designed to supplement T-cell response data for the patient or patient population.

(17) The method of any of (1)-(16), wherein the method further comprises using the TCR assay to classify or estimate a patient state.

(18) The method of (17), wherein the method further comprises administering a therapeutic treatment to a patient based on the classified or estimated patient state.

(19) A computer program product comprising a non-transitory computer readable medium comprising processor-executable instructions that, when executed by one or more processors, cause the one or more processors to perform operations to:
obtain a first plurality of inputs representing a plurality of peptides;
train an Artificial Neural Network (ANN) defining a pan-human leukocyte antigen (HLA) binding classifier model using the first plurality of inputs, wherein the trained HLA binding classifier model is configured to determine average binding predictions of overlapping peptides at each position of the viral or cancer protein independently for each of a plurality of test HLAs comprising HLA-I and HLA-II functional groupings;
obtain a second plurality of inputs representing a viral or cancer protein encoded into a plurality of peptides;
feed the second plurality of inputs into the trained HLA binding classifier model, wherein the trained HLA binding classifier is configured to determine average binding predictions of overlapping peptides of the plurality of peptides;
select, based on the average binding predictions, one or more peptide pools from the plurality of peptides;
obtain a third plurality of inputs associated with a plurality of blood samples, wherein the blood samples are representative of a patient or patient population;
instantiate, based on the one or more peptide pools and the third plurality of inputs, a T-cell response model; wherein the T-cell response model is trained to predict peptides and protein fragments associated with a high probability of eliciting T-cell response, based on validated T-cell epitopes and peptides failing to elicit a T-cell response;
sequence, by a sequencer, responding T-cells identified based on T-cell response criteria;
detect, based on data obtained from sequencing the responding T-cells, one or more T-cell response patterns common to the patient or patient population;
train a TCR classifier/regression model to predict or estimate a patient state using datasets based on the one or more T-cell response patterns;
determining, using the trained TCR classifier/regression model, a minimum set of T-cell receptors for classifying or estimating the patient state; and
designing one or more primers based on the determined minimum set of T-cell receptors, the one or more primers defining a TCR assay for classifying or estimating the patient state.

(20) A computer system comprising:
a memory storing one or more instructions for designing a T-cell receptor (TCR) assay; and
one or more processors, coupled with the memory, the one or more processors configured to execute the one or more instructions to perform operations to: obtain a first plurality of inputs representing a plurality of peptides; train an Artificial Neural Network (ANN) defining a pan-human leukocyte antigen (HLA) binding classifier model using the first plurality of inputs, wherein the trained HLA binding classifier model is configured to determine average binding predictions of overlapping peptides at each position of the viral or cancer protein independently for each of a plurality of test HLAs comprising HLA-I and HLA-II functional groupings;
obtain a second plurality of inputs representing a viral or cancer protein encoded into a plurality of peptides;
feed the second plurality of inputs into the trained HLA binding classifier model, wherein the trained HLA binding classifier is configured to determine average binding predictions of overlapping peptides of the plurality of peptides;
select, based on the average binding predictions, one or more peptide pools from the plurality of peptides;
obtain a third plurality of inputs associated with a plurality of blood samples, wherein the blood samples are representative of a patient or patient population;
instantiate, based on the one or more peptide pools and the third plurality of inputs, a T-cell response model; wherein the T-cell response model is trained to predict peptides and protein fragments associated with a high probability of eliciting T-cell response, based on validated T-cell epitopes and peptides failing to elicit a T-cell response;
sequence, by a sequencer, responding T-cells identified based on T-cell response criteria;
detect, based on data obtained from sequencing the responding T-cells, one or more T-cell response patterns common to the patient or patient population;
train a TCR classifier/regression model to predict or estimate a patient state using datasets based on the one or more T-cell response patterns;
determine, using the trained TCR classifier/regression model, a minimum set of T-cell receptors for classifying or estimating the patient state; and
design one or more primers based on the determined minimum set of T-cell receptors, the one or more primers defining a TCR assay for classifying or estimating the patient state.

It should be appreciated that the methods described herein may be performed with a digital processing system, such as a conventional, general-purpose computer system. Special purpose computers, which are designed or programmed to perform only one function may be used in the alternative. FIG. 5 is an illustration showing an exemplary computing device which may implement the embodiments described herein. The computing device of FIG. 5 may be used to perform embodiments of the functionality for performing the designing of TCR assays in accordance with some embodiments. The computing device includes central processing unit (CPU) 502, which is coupled via bus 506 to memory 504 and mass storage device 508. Mass storage device 508 represents a persistent data storage device such as a floppy disc drive or a fixed disc drive, which may be local or remote in some embodiments. Mass storage device 508 may be implemented as a backup storage, in some embodiments. Memory 504 may include read only memory, random access memory, etc. Applications resident on the computing device may be stored on or accessed through a computer readable medium such as memory 504 or mass storage device 508 in some embodiments. Applications may also be in the form of modulated electronic signals modulated accessed through a network modem or other network interface of the computing device. It should be appreciated that CPU 502 may be embodied in a general-purpose processor, a special purpose processor, or a specially programmed logic device in some embodiments.

Display 512 is in communication with CPU 502, memory 504, and mass storage device 508, through bus 506. Display 512 is configured to display any visualization tools or reports associated with the system described herein. Input/output device 510 is coupled to bus 506 in order to communicate information in command selections to CPU 502. It should be appreciated that data to and from external devices may be communicated through the input/output device 510. CPU 502 can be defined to execute the functionality described herein to enable the functionality described with reference to FIGS. 1-4. The code embodying this functionality may be stored within memory 504 or mass storage device 508 for execution by a processor such as CPU 502 in some embodiments. The operating system on the computing device may be iOS™, MS-WINDOWS™, OS/2™, UNIX™, LINUX™, or other known operating systems. It should also be appreciated that the embodiments described herein may be integrated with virtualized computing system.

In the above description, numerous details are set forth. It will be apparent, however, to one skilled in the art, that the present invention may be practiced without these specific details. In some instances, well-known structures and devices are shown in block diagram form, rather than in detail, in order to avoid obscuring the present invention.

It is to be understood that the above description is intended to be illustrative, and not restrictive. Many other embodiments will be apparent to those of skill in the art upon reading and understanding the above description. Although the present invention has been described with reference to specific exemplary embodiments, it will be recognized that the invention is not limited to the embodiments described, but can be practiced with modification and alteration within the spirit and scope of the appended claims. Accordingly, the specification and drawings are to be regarded in an illustrative sense rather than a restrictive sense. The scope of the invention should, therefore, be determined with reference to the appended claims, along with the full scope of equivalents to which such claims are entitled.

It should be understood that although the terms first, second, etc. may be used herein to describe various steps or calculations, these steps or calculations should not be limited by these terms. These terms are only used to distinguish one step or calculation from another. For example, a first calculation could be termed a second calculation, and, similarly, a second step could be termed a first step, without departing from the scope of this disclosure. As used herein, the term “and/or” and the “I” symbol includes any and all combinations of one or more of the associated listed items. As used herein, the singular forms “a”, “an” and “the” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

Although the method operations were described in a specific order, it should be understood that other operations may be performed in between described operations, described operations may be adjusted so that they occur at slightly different times or the described operations may be distributed in a system which allows the occurrence of the processing operations at various intervals associated with the processing.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved. With the above embodiments in mind, it should be understood that the embodiments might employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing. Any of the operations described herein that form part of the embodiments are useful machine operations. The embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines can be used with computer programs written in accordance with the teachings herein, or it may be more convenient to construct a more specialized apparatus to perform the required operations.

A module, an application, a layer, an agent or other method-operable entity could be implemented as hardware, firmware, or a processor executing software, or combinations thereof. It should be appreciated that, where a software-based embodiment is disclosed herein, the software can be embodied in a physical machine such as a controller. For example, a controller could include a first module and a second module. A controller could be configured to perform various actions, e.g., of a method, an application, a layer or an agent.

The embodiments can also be embodied as computer readable code on a non-transitory computer readable medium. The computer readable medium is any data storage device that can store data, which can be thereafter read by a computer system. Examples of the computer readable medium include hard drives, network attached storage (NAS), read-only memory, random-access memory, CD-ROMs, CD-Rs, CD-RWs, magnetic tapes, flash memory devices, and other optical and non-optical data storage devices. The computer readable medium can also be distributed over a network coupled computer system so that the computer readable code is stored and executed in a distributed fashion. Embodiments described herein may be practiced with various computer system configurations including hand-held devices, tablets, microprocessor systems, microprocessor-based or programmable consumer electronics, minicomputers, mainframe computers and the like. The embodiments can also be practiced in distributed computing environments where tasks are performed by remote processing devices that are linked through a wire-based or wireless network.

In various embodiments, one or more portions of the methods and mechanisms described herein may form part of a cloud-computing environment. In such embodiments, resources may be provided over the Internet as services according to one or more various models. Such models may include Infrastructure as a Service (IaaS), Platform as a Service (PaaS), and Software as a Service (SaaS). In IaaS, computer infrastructure is delivered as a service. In such a case, the computing equipment is generally owned and operated by the service provider. In the PaaS model, software tools and underlying equipment used by developers to develop software solutions may be provided as a service and hosted by the service provider. SaaS typically includes a service provider licensing software as a service on demand. The service provider may host the software, or may deploy the software to a customer for a given period of time. Numerous combinations of the above models are possible and are contemplated.

Various units, circuits, or other components may be described or claimed as “configured to” perform a task or tasks. In such contexts, the phrase “configured to” is used to connote such structure by indicating that the units/circuits/components include structure (e.g., circuitry) that performs the task or tasks during operation. As such, the unit/circuit/component can be said to be configured to perform the task even when the specified unit/circuit/component is not currently operational (e.g., is not on). The units/circuits/components used with the “configured to” language include hardware; for example, circuits, memory storing program instructions executable to implement the operation, etc. Reciting that a unit/circuit/component is “configured to” perform one or more tasks is expressly intended not to invoke 35 U.S.C. 112, sixth paragraph, for that unit/circuit/component. Additionally, “configured to” can include generic structure (e.g., generic circuitry) that is manipulated by software and/or firmware (e.g., an FPGA or a general-purpose processor executing software) to operate in manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks.

What is claimed is:

1. A method of designing a T-cell receptor (TCR) assay performed by a processor-based TCR assay module, the method comprising:
obtaining a first plurality of inputs representing a plurality of peptides;
training an Artificial Neural Network (ANN) defining a pan-human leukocyte antigen (HLA) binding classifier model using the first plurality of inputs, wherein the trained HLA binding classifier model is configured to determine average binding predictions of overlapping peptides at each position of the viral or cancer protein independently for each of a plurality of test HLAs comprising HLA-I and HLA-II functional groupings;
obtaining a second plurality of inputs representing a viral or cancer protein encoded into a plurality of peptides;
feeding the second plurality of inputs into the trained HLA binding classifier model, wherein the trained HLA binding classifier is configured to determine average binding predictions of overlapping peptides of the plurality of peptides;
selecting, based on the average binding predictions, one or more peptide pools from the plurality of peptides;
obtaining a third plurality of inputs associated with a plurality of blood samples, wherein the blood samples are representative of a patient or patient population;
instantiating, based on the one or more peptide pools and the third plurality of inputs, a T-cell response model; wherein the T-cell response model is trained to predict peptides and protein fragments associated with a high probability of eliciting T-cell response, based on validated T-cell epitopes and peptides failing to elicit a T-cell response;
sequencing, by a sequencer, responding T-cells identified based on T-cell response criteria;
detecting, based on data obtained from sequencing the responding T-cells, one or more T-cell response patterns common to the patient or patient population;
training a TCR classifier/regression model to predict or estimate a patient state using datasets based on the one or more T-cell response patterns;
determining, using the trained TCR classifier/regression model, a minimum set of T-cell receptors for classifying or estimating the patient state; and
designing one or more primers based on the determined minimum set of T-cell receptors, the one or more primers defining a TCR assay for classifying or estimating the patient state.

2. The method of claim 1, wherein the training of the HLA binding classifier model comprises,
obtaining a plurality of test HLAs encoded into variable-length proteins, wherein the plurality of test HLAs comprises HLA-I and HLA-II functional groupings;
processing the encoded variable-length peptides corresponding to the viral protein and the variable-length proteins corresponding to the plurality of test HLAs using the classifier model such that, independently per test HLA, the classifier model is operable to determine an average binding prediction of overlapping peptides at each position of the viral protein;
independently per test HLA:
mapping in aggregate average binding predictions to locations along the test viral protein such that peptide-HLA interaction is indicated;
determining nearest max locations for the average binding predictions using a sliding window having a fixed length;
determining top max regions by selecting the nearest max locations having average binding predictions within a top percentage of values;
selecting peptides classified as binders that overlap the top max regions; and
determining a pan-HLA max region, wherein the determining includes setting unselected locations to zero, calculating a mean along an HLA axis of the average binding prediction, and selecting pan-HLA maxima within a top percentage of values based on the mean;
independently for each of the HLA-I and HLA-II functional groupings:
filtering the selected peptides classified as binders to identify candidate peptides that overlap the top max regions based on an aggregate of the pan-HLA max regions; and
including one or more of the candidate peptides in an mRNA-based vaccine or therapeutic treatment for a patient.

3. The method of claim 1, wherein the training of the TCR classifier/regression model to predict or estimate a patient state comprises:
differentiating TCR sequences specific to a patient state from general TCR sequences associated with patients not representative of a condition of interest associated with the patient state;
identifying T-cell response patterns based on the differentiation; and
generating the TCR classifier/regression model based on the identified T-cell response patterns.

4. The method of claim 1, wherein the training of the TCR classifier/regression model to predict or estimate a patient state comprises:
differentiating TCR sequences specific to a patient state from general TCR sequences associated with patients not representative of a condition of interest associated with the patient state;
identifying, based on the differentiation, TCR sequences in a sequence embedding space associated with the condition of interest; and
generating the TCR classifier/regression model with the identified TCR sequences.

5. The method of claim 1, wherein the determining of the minimum set of T-cell receptors comprises:
retrieving prediction scores of the trained TCR classifier/regression model for a plurality of patients; and
selecting, based on the retrieved prediction scores, one or more TCR patterns.

6. The method of claim 1, wherein the method further comprises selecting the one or more peptide pools based on one or more of: a specific site, a hotspot, or a receptor-binding domain of the viral or cancer protein.

7. The method of claim 1, wherein the method further comprises selecting the one or more peptide pools based on multiple regions or hotspots of the viral or cancer protein.

8. The method of claim 1, wherein the method further comprises selecting the one or more peptide pools based on an entire viral or cancer protein.

9. The method of claim 1, wherein the method further comprises selecting the one or more peptide pools based on at least one of CD4 T-cell interaction or CD8 T-cell interaction.

10. The method of claim 1, wherein the method further comprises selecting the one or more peptide pools based on the average binding predictions for the HLA-I functional groupings.

11. The method of claim 1, wherein the method further comprises selecting the one or more peptide pools based on the average binding predictions for the HLA-II functional groupings.

12. The method of claim 1, wherein the method further comprises selecting the one or more peptide pools based on areas of predicted binding frequency across the HLA-I and HLA-II functional groupings.

13. The method of claim 1, wherein the method further comprises selecting the one or more peptide pools based on a pan-HLA binding prediction.

14. The method of claim 1, wherein the test for T cell response comprises at least one of the following: an enzyme-linked immunosorbent spot (ELISpot) assay test, a cytotoxic T Lymphocyte (CTL) assay test, and a DNA barcoded peptide-MHC (pMHC) multimers test.

15. The method of claim 1, wherein the test for T-cell response further comprises testing a synthetic TCR assay for T-cell response.

16. The method of claim 15, wherein the synthetic TCR assay is designed to supplement T-cell response data for the patient or patient population.

17. The method of claim 1, wherein the method further comprises using the TCR assay to classify or estimate a patient state.

18. The method of claim 17, wherein the method further comprises administering a therapeutic treatment to a patient based on the classified or estimated patient state.

19. A computer program product comprising a non-transitory computer readable medium comprising processor-executable instructions that, when executed by one or more processors, cause the one or more processors to perform operations to:
obtain a first plurality of inputs representing a plurality of peptides;
train an Artificial Neural Network (ANN) defining a pan-human leukocyte antigen (HLA) binding classifier model using the first plurality of inputs, wherein the trained HLA binding classifier model is configured to determine average binding predictions of overlapping peptides at each position of the viral or cancer protein independently for each of a plurality of test HLAs comprising HLA-I and HLA-II functional groupings;
obtain a second plurality of inputs representing a viral or cancer protein encoded into a plurality of peptides;
feed the second plurality of inputs into the trained HLA binding classifier model, wherein the trained HLA binding classifier is configured to determine average binding predictions of overlapping peptides of the plurality of peptides;
select, based on the average binding predictions, one or more peptide pools from the plurality of peptides;
obtain a third plurality of inputs associated with a plurality of blood samples, wherein the blood samples are representative of a patient or patient population;
instantiate, based on the one or more peptide pools and the third plurality of inputs, a T-cell response model; wherein the T-cell response model is trained to predict peptides and protein fragments associated with a high probability of eliciting T-cell response, based on validated T-cell epitopes and peptides failing to elicit a T-cell response;
sequence, by a sequencer, responding T-cells identified based on T-cell response criteria;
detect, based on data obtained from sequencing the responding T-cells, one or more T-cell response patterns common to the patient or patient population;
train a TCR classifier/regression model to predict or estimate a patient state using datasets based on the one or more T-cell response patterns;
determining, using the trained TCR classifier/regression model, a minimum set of T-cell receptors for classifying or estimating the patient state; and
designing one or more primers based on the determined minimum set of T-cell receptors, the one or more primers defining a TCR assay for classifying or estimating the patient state.

20. A computer system comprising:
a memory storing one or more instructions for designing a T-cell receptor (TCR) assay; and
one or more processors, coupled with the memory, the one or more processors configured to execute the one or more instructions to perform operations to:
obtain a first plurality of inputs representing a plurality of peptides;
train an Artificial Neural Network (ANN) defining a pan-human leukocyte antigen (HLA) binding classifier model using the first plurality of inputs, wherein the trained HLA binding classifier model is configured to determine average binding predictions of overlapping peptides at each position of the viral or cancer protein independently for each of a plurality of test HLAs comprising HLA-I and HLA-II functional groupings;
obtain a second plurality of inputs representing a viral or cancer protein encoded into a plurality of peptides;
feed the second plurality of inputs into the trained HLA binding classifier model, wherein the trained HLA binding classifier is configured to determine average binding predictions of overlapping peptides of the plurality of peptides;
select, based on the average binding predictions, one or more peptide pools from the plurality of peptides;
obtain a third plurality of inputs associated with a plurality of blood samples, wherein the blood samples are representative of a patient or patient population;
instantiate, based on the one or more peptide pools and the third plurality of inputs, a T-cell response model; wherein the T-cell response model is trained to predict peptides and protein fragments associated with a high probability of eliciting T-cell response, based on validated T-cell epitopes and peptides failing to elicit a T-cell response;
sequence, by a sequencer, responding T-cells identified based on T-cell response criteria;
detect, based on data obtained from sequencing the responding T-cells, one or more T-cell response patterns common to the patient or patient population;
train a TCR classifier/regression model to predict or estimate a patient state using datasets based on the one or more T-cell response patterns;
determine, using the trained TCR classifier/regression model, a minimum set of T-cell receptors for classifying or estimating the patient state; and
design one or more primers based on the determined minimum set of T-cell receptors, the one or more primers defining a TCR assay for classifying or estimating the patient state.
